(19) United States
(12) Patent Application Publication
(10) Pub. No.: US 2022/0117919 A1
(43) Pub. Date: Apr. 21, 2022

(54) ENHANCED BIOAVAILABILITY OF N-(2,6-BIS(1-METHYLETHYL)PHENYL)-N'-((1-(4-(DIMETHYLAMINO)-PHENYL)CYCLOPENTYL)METHYL)UREA HYDROCHLORIDE

(71) Applicant: The Regents of the University of Michigan, Ann Arbor, MI (US)

(72) Inventor: Pharis MOHIDEEN, Pine Brook, NJ (US)

(73) Assignee: The Regents of the University of Michigan, Ann Arbor, MI (US)

(21) Appl. No.: 17/514,538

(22) Filed: Oct. 29, 2021

Related U.S. Application Data
(63) Continuation of application No. 17/191,556, filed on Mar. 3, 2021, now abandoned.
Application No. 17/191,556 is a continuation of application No. 16/936,279, filed on Jul. 22, 2020, now abandoned.
Application No. 16/936,279 is a continuation of application No. 16/702,322, filed on Dec. 3, 2019, now abandoned.
Application No. 16/702,322 is a continuation of application No. 15/743,418, filed on Jan. 10, 2018, now abandoned.
(60) Provisional application No. 62/191,195, filed on Jul. 10, 2015.

(51) Int. Cl. A61K 31/17; A61K 9/20; A61P 35/00; A61K 9/28; A61K 31/136

(52) U.S. Cl. CPC A61K 31/17; A61K 9/2054; A61K 9/2018; A61K 9/28; A61K 31/136; A61P 35/00

(57) ABSTRACT

Methods for enhancing the bioavailability of N-(2,6-bis(1-methylethyl)phenyl)-N′-((1-(4-(dimethylamino)phenyl)cyclopentyl)-methyl)urea hydrochloride (ATR-101) through administration with food, and compositions and kits related thereto.

### BACKGROUND

### Technical Field

This invention is directed to methods for enhancing the bioavailability of N-(2,6-bis(1-methylethyl)phenyl)-N′-((1-(4-(dimethylamino)-phenyl)cyclopentyl)methyl)urea hydrochloride (referred to herein as “ATR-101”), as well as to compositions and kits related to the same.

### Description of the Related Art

The adrenal gland is made up of two parts: the outer cortex in which certain hormones are produced, and the inner medulla which is part of the nervous system, wherein nervous system hormones are produced. The cortex is devoted to the synthesis of glucocorticoid, mineralocorticoid and androgen hormones. Specific cortical cells produce particular hormones including aldosterone, cortisol, and androgens such as androstenedione. Adrenocortical tumors originate in the cortex.

There are two main types of adrenal cortex tumors: adenomas which are benign and adrenocortical carcinoma which are malignant. Adenomas in many people produce no symptoms, but in some instances the tumors lead to excess hormone production. Adrenocortical carcinoma can produce the hormones cortisol, aldosterone, estrogen, or testosterone, as well as other hormones. Adrenocortical carcinomas (ACC) are rare, highly malignant tumors. In women, if the tumor releases these hormones, it can lead to male characteristics. The excess hormones may or may not cause symptoms. In general, adenomas are treated by surgical removal or with therapeutic intervention. Likewise, adrenocortical carcinomas can lead to hormone production that can cause noticeable body changes such as weight gain, fluid build-up, early puberty in children, or excess facial or body hair in women. While the cause is unknown, adrenocortical carcinoma is most common in children younger than 5 and adults in their 30s and 40s. Adrenocortical carcinoma may be linked to a cancer syndrome that is passed down through families (inherited). Both men and women can develop this tumor.

While the understanding of the disease has advanced with the advent of modern molecular techniques, the prognosis of patients with advanced disease, who represent more than half of the diagnoses, remains dismal. (Hammer, G. D. and T. Else, eds., Adrenocortical Carcinoma, Basic Science and Clinical Concepts, 2011, New York: Springer).

The sole FDA-approved therapeutic agent for ACC is mitotane (o,p′-DDD), a derivative of the insecticide DDT, discovered in 1950s, when it was found to destroy the adrenal cortex of dogs. Despite half a century of use, its molecular mechanism remains unclear. The drug requires chemical transformation into an active, free radical form, which then induces lipid peroxidation and cell death. Mitotane also suppresses steroidogenesis and inhibits other cytochrome P450-class enzymes (Id.).

Whereas mitotane is widely used for the treatment of ACC, it has increased progression-free survival in only one-quarter to one-third of patients. For the patients that derive a therapeutic benefit, the effect is transient, delaying disease progression by an average of five months (Id.). Mitotane has numerous problems as a therapeutic agent, making its use difficult, and requiring close monitoring of patients.

Accordingly, there remains a significant need for new therapeutic agents useful for treatment of ACC and other related diseases or conditions. One such promising agent is N-(2,6-bis(1-methylethyl)phenyl)-N′-((1-(4-(dimethyl-amino)phenyl)cyclopentyl)-methyl)urea hydrochloride (“ATR-101”) as disclosed in Published U.S. Patent Application US 2013/0267550 A1 entitled “Compounds and Methods for Treating Aberrant Adrenocortical Cell Disorders” (which application is hereby incorporated by reference in its entirety). The free base form of ATR-101 has the following chemical structure:

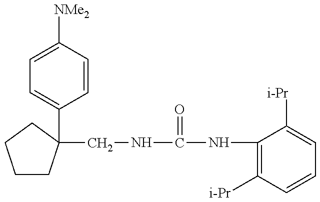 [SMILES: CC(C)c1cccc(C(C)C)c1NC(=O)NCC1(c2ccc(N(C)C)cc2)CCCC1]

While significant advances have been made in this field, particularly in the context of ATR-101, there remains a substantial need for improved techniques and products to increase the bioavailability of ATR-101 to patients in need thereof, including patients having ACC and/or other disorders or conditions.

### BRIEF SUMMARY

This present invention generally provides methods for enhancing the bioavailability of N-(2,6-bis(1-methylethyl)phenyl)-N′-((1-(4-(dimethylamino)-phenyl)cyclopentyl)methyl)urea hydrochloride (referred to herein as “ATR-101”) as well as to compositions and kits relating to the administration thereof, particularly in the context of a solid oral drug form ATR-101.

In one embodiment, a method is provided to increase the bioavailability of ATR-101 by orally administering to a subject in need thereof ATR-101 in unit dosage form at or near the time of oral administration of food. In even more specific embodiments, co-administration comprises eating all or a portion of meal followed by oral administration of the solid pharmaceutical composition. Suitable food in this context includes any food product with caloric content, including both solid and liquid forms.

In one embodiment, a solid pharmaceutical composition is provided in a unit dosage form suitable for oral administration. The composition comprises ATR-101 in combination with one or more pharmaceutically acceptable carriers or excipients, wherein ATR-101 is present in the solid pharmaceutical composition in the unit dosage form at a level ranging from about 250-750 mg as measured as the free base form of ATR-101. In more specific embodiments, ATR-101 is present in the solid pharmaceutical composition at a level at or in excess of 50%, 60%, 65% or 70% by weight, as measured as the free base form of ATR-101, of the total weight of the unit dosage form.

In another embodiment, a method is provided for administering a solid pharmaceutical composition, comprising orally administering to a subject in need thereof ATR-101 in unit dosage form. In a more specific embodiment, such methods comprise oral administration of food at or near the time of oral administration of ATR-101 in unit dosage form. In even more specific embodiments, co-administration comprises eating all or a portion of a meal followed by oral administration of the solid pharmaceutical composition.

In a further embodiment, a kit is provided for co-administration of ATR-101 with food. Such kits comprise a plurality of oral unit dosage forms of ATR-101 in combination with instructions for co-administration with food, at or near the time of oral administration of ATR-101 in unit dosage form.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

FIG. 1 is a mean plasma concentration of ATR-101 versus time graph following single oral administration of 500 mg ATR-101 under the treatment protocols labelled Treatment A, B, and C in Example 3.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, the term “about” as used herein means ±20% of the stated value, and in more specific embodiments means±10%, ±5%, ±2% and ±1% of the stated value.

As used herein, “ATR-101” refers to N-(2,6-bis(1-methylethyl)-phenyl)-N′-((1-(4-(dimethylamino)-phenyl)cyclopentyl)methyl)urea hydrochloride. While ATR-101 refers to the hydrochloride salt form of this compound, it should be understood that other pharmaceutically acceptable salts may be prepared and that such salt forms are within the scope of this invention.

As used herein, “administered with food” refers to administration with any food product, whether in solid or liquid form, with caloric content. Preferably the food is a solid food with sufficient bulk and fat content that it is not rapidly dissolved and absorbed in the stomach. More preferably the food is a meal, such as breakfast, lunch or dinner. The dosage of the ATR-101 may be administered to the subject, for example, between about 30 minutes prior to about 2 hours after eating a meal, most advantageously the dosage is administered within 15 minutes after eating a meal. The terms “without food”, “fasted” and “an empty stomach” refer to the condition of not having consumed food for about 2 hours prior to until about 1 hour after administration.

As used herein, “high-fat” refers to any food product, solid or liquid, with approximately 50 percent of total caloric content coming from fat. As used herein, “high-calorie meal” refers to any meal having approximately 800 to 1000 calories. A representative high-fat, high-calorie meal should derive approximately 150, 250, and 500-600 calories from protein, carbohydrate and fat, respectively.

As used herein, “aberrant adrenocortical cellular behavior” includes increased hormone production, Cushing's syndrome, benign adenoma, adrenocortical carcinoma (ACC), metastatic adrenocortical carcinoma, congenital adrenal hyperplasia, hyperaldosteronism including Conn syndrome, a unilateral aldosterone-producing adenoma, bilateral adrenal hyperplasia (or idiopathic hyperaldosteronism (IHA)), renin-responsive adenoma, primary adrenal hyperplasia and glucocorticoid-remediable aldosteronism (GRA), and 21-hydroxylase deficiency.

As used herein, “disorders associated with aberrant adrenocortical cellular behavior” is used herein to mean symptoms and/or conditions that arise, either directly or indirectly, from aberrant adrenocortical cellular behavior. As will become apparent herein, these symptoms and/or conditions that arise, either directly or indirectly, from aberrant adrenocortical cellular behavior are numerous. As used herein, “adrenocortical” and “adrenal cortex” are intended to mean the same.

As used herein, “Cushing's syndrome” means a hormonal disorder caused by prolonged exposure of the body's tissues to high levels of cortisol. Cushing's syndrome is sometimes referred to as “hypercortisolism” (excess cortisol production). Cushing's syndrome includes various subtypes of the disease, including Cushing's disease, adrenal Cushing's syndrome, and ectopic ACTH syndrome, which are categorized by the cause of hypercortisolism. Cushing's disease, also known as pituitary Cushing's, is caused by a pituitary gland tumor which secretes excessive ACTH, which in turn stimulates the adrenal glands to make more cortisol. Ectopic ACTH syndrome is caused by tumors that arise outside the pituitary gland that can produce ACTH, which stimulates cortisol production. Adrenal Cushing's syndrome is caused by an abnormality of the adrenal gland, usually an adrenal tumor, which causes excess cortisol secretion.

As used herein, “subject” means a mammal, including a human.

As used herein, the phrase term “therapeutically effective amount” refers to an amount of a therapeutic agent to treat, ameliorate, or prevent a disease or condition, or to exhibit a detectable therapeutic or preventative effect. The effect is detected by, for example, a reduction in tumor size. The effect is also detected by, for example, chemical markers, steroid levels, or antigen levels. Therapeutic effects also include reduction in physical symptoms, such as decreased body temperature. The precise effective amount for a subject will depend upon the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “treatment” includes therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

In one embodiment, the disorder is associated with aberrant adrenocortical cellular behavior. Thus, in this context, “treatment” includes therapeutic applications to slow or stop progression of a disorder associated with aberrant adrenocortical cellular behavior, prophylactic application to prevent development of a disorder associated with aberrant adrenocortical cellular behavior, and reversal of a disorder associated with aberrant adrenocortical cellular behavior. In this context, reversal of a disorder differs from a therapeutic application which slows or stops a disorder in that with a method of reversing, not only is progression of a disorder completely stopped, cellular behavior is moved to some degree, toward a normal state that would be observed in the absence of aberrant adrenocortical cellular behavior.

In another embodiment, the disorder is one that would benefit from inhibition of acyl-coenzyme A:cholesterol transferase (ACAT). To this end, ATR-101 is an ACAT inhibitor. ACAT is an integral membrane protein localized in the endoplasmic reticulum and catalyzes formation of cholesteryl esters (CE) (also known as cholesterol esters) from cholesterol and fatty acyl coenzyme A. Cholesteryl esters are stored as cytoplasmic lipid droplets in the cell. In mammals, there are two ACAT isoenzymes, ACAT1 and ACAT2. ACAT2 is expressed in the liver and intestine. ACAT1 expression is more ubiquitous and is present in cells and tissues such as macrophages, adrenal glands, hepatocytes, enterocytes, renal tubule cells, and neurons.

Altered lipid metabolism has been identified as an important process in cancer. For example, aberrant cholesteryl ester accumulation has been found in advanced prostate cancers, and inhibition of cholesterol esterification has been reported to impair cancer aggressiveness (Yue et al. “Cholesteryl ester accumulation induced by PTEN loss and PI3K/AKT activation underlies human prostate cancer aggressiveness,” Cell Metab. 4:393-406, 2014). Accordingly, in this embodiment, “treatment” includes therapeutic applications to slow or stop progression of, prophylactic application to prevent development of, or reversal of, a disorder that would benefit from inhibition of ACAT, including disorders that would benefit from inhibition of cholesterol esterification. Representative disorders in this regard include cancers in which aberrant cholesteryl ester accumulation are present, and/or in which cholesterol esterification impairs cancer growth or aggressiveness. Representative cancers in this regards include (but are not limited to) prostate cancer and ovarian cancer.

In one embodiment, the solid pharmaceutical composition is co-administered with food to increase exposure of ATR-101 upon oral administration. Such food products have surprisingly been found to significantly increase exposure of ATR-101 upon oral administration. In more specific embodiments, co-administration comprises eating food simultaneously with, or in close proximity to, oral administration of the solid pharmaceutical composition.

In some embodiments, the co-administered food is a high-fat, high calorie meal. Representative high-fat meals have approximately 50 percent of total caloric content of the meal coming from fat and representative high-calorie meals have approximately 800 to 1000 calories. A representative meal should derive approximately 150, 250, and 500-600 calories from protein, carbohydrate, and fat, respectively. The amount of food co-administration with the solid pharmaceutical composition should be sufficient such that increased exposure of ATR-101 is achieved upon oral administration.

In one embodiment, the mean maximum plasma concentration (Cₘₐₓ) of ATR-101 is increased when ATR-101 is administered with a high-fat, high calorie meal, compared to when ATR-101 is administered under fasting conditions. In a specific embodiment, Cₘₐₓ increases by at least about 50%, 60%, 70%, 80%, 90%, 100%, 110%, 120%, 130%, 140%, 150%, 160%, 170%, 180%, 190%, or 200% when ATR-101 is administered with a high-fat, high calorie meal, compared to when ATR-101 is administered under fasting conditions.

In one embodiment, the area under the plasma concentration time curve (AUC₀₋ₜ) of ATR-101 is increased when ATR-101 is administered with a high-fat, high calorie meal, compared to when ATR-101 is administered under fasting conditions. In a specific embodiment, AUC₀₋ₜ increases by at least about 50%, 60%, 70%, 80%, 90%, 100%, 110%, 120%, 130%, 140%, 150%, 160%, 170%, 180%, 190%, or 200% when ATR-101 is administered with a high-fat, high calorie meal, compared to when ATR-101 is administered under fasting conditions.

In one embodiment, a solid pharmaceutical composition is provided in a unit dosage form suitable for oral administration. The composition comprises ATR-101 in combination with one or more pharmaceutically acceptable carriers or excipients, wherein ATR-101 is present in the solid pharmaceutical composition in the unit dosage form at a level ranging from about 250-750 mg as measured as the free base form of ATR-101. More specific representative levels include, 250 mg, 300 mg, 350 mg, 400 mg, 450 mg, 500 mg, 550 mg, 600 mg, 650 mg, 700 mg and 750 mg, as measured as the free base form of ATR-101.

In more specific embodiments, ATR-101 is present in the solid pharmaceutical composition at a level at or in excess of 50% by weight, as measured as the free base form of ATR-101, of the total weight of the unit dosage form. In a further embodiment, ATR-101 is present in the solid pharmaceutical composition at a level at or in excess of 60% by weight, as measured as the free base form of ATR-101, of the total weight of the unit dosage form. In a further embodiment, ATR-101 is present in the solid pharmaceutical composition at a level at or in excess of 65% by weight, as measured as the free base form of ATR-101, of the total weight of the unit dosage form. In still a further embodiment, ATR-101 is present in the solid pharmaceutical composition at a level at or in excess of 70% by weight, as measured as the free base form of ATR-101, of the total weight of the unit dosage form. Surprisingly, the nature of ATR-101 itself, including particle size distribution, bulk density, compressibility and the like, allows for such elevated drug levels within the solid pharmaceutical composition.

ATR-101 present within the solid pharmaceutical composition may be characterized by the median diameter of its particles, as determined in dry-dispersion mode using a Malvern Mastersizer 2000 equipped with a Scirocco 2000 module (measuring range 0.02 to 2000 μm). For example, the “d(0.5)” particle size distribution is the median diameter of the particle size distribution, and represents the particle size at which 50% of the particles are larger and 50% of the particles are smaller than the d(0.5) value. Similarly, the “d(0.1)” value is the particle size at which 10% of the particles are smaller and 90% of the particles are larger, and the “d(0.9)” value is the particle size at which 90% of the particles are smaller and 10% of the particles are larger.

In one embodiment, ATR-101 of the solid pharmaceutical composition has a d(0.5) particle size distribution of about 12 μm, and can be further characterized as having a d(0.1) particle size distribution of about 2 μm, and/or as having a d(0.9) particle size distribution of about 49 μm. In other embodiments, ATR-101 of the solid pharmaceutical composition has a d(0.5) particle size distribution ranging from 5 to 20 μm, from 6 to 18 μm, from 8 to 16 μm, from 10 to 14 μm, or from 2 to 10 μm. In still other embodiments, ATR-101 of the solid pharmaceutical composition has a d(0.1) particle size distribution greater than 1 μm and/or a d(0.9) particle size distribution less than 60 μm or, in another embodiment, less than 50 μm.

The solid pharmaceutical composition may be in a variety of forms suitable to oral administration, such as a capsule, pill, granule, suspension, pellet, tablet or powder, and contains one or more pharmaceutically acceptable carriers or excipients. In a specific embodiment, the solid pharmaceutical composition is in the form of a compressed tablet, which may be coated, for example with a nonfunctional film or a release-modifying coating. Pharmaceutically acceptable carriers and excipients are well known in the art, such as those disclosed in Remington: The Science and Practice of Pharmacy, 22^{nd} Edition, Allen, Lloyd V., Jr. Ed. (2012) (incorporated herein by reference), and include (without limitation) diluents, binding agents, adhesives, disintegrants, wetting agents, lubricant, anti-adherent, glidant, tonicity agent and/or surfactant.

In various aspects, the solid pharmaceutical composition includes a diluent, either individually or in combination, such as, and without limitation, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. The diluent or diluents selected should exhibit suitable flow properties and, where tablets are desired, compressibility.

In various aspects, the solid pharmaceutical composition includes binding agents or adhesives which are useful excipients, particularly where the composition is in the form of a tablet. Such binding agents and adhesives should impart sufficient cohesion to the blend being formulated in a tablet to allow for normal processing operations such as sizing, lubrication, compression and packaging, but still allow the tablet to disintegrate and the compound to be absorbed upon ingestion. Suitable binding agents and adhesives include, either individually or in combination, acacia; tragacanth; glucose; polydextrose; starch including pregelatinized starch;

gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, constitute in various aspects, in total about 0.5% to about 25%, for example about 0.75% to about 15%, or about 1% to about 10%, by weight of the composition.

In various aspects, the solid pharmaceutical composition includes a disintegrant. Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

In various aspects, the solid pharmaceutical composition includes a wetting agent. Wetting agents, if present, are normally selected to maintain the compound in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laureate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

In various aspects, the solid pharmaceutical composition includes a lubricant. Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

In various aspects, the solid pharmaceutical composition includes an anti-adherent. Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

In various aspects, the solid pharmaceutical composition includes a glidant. Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

In various aspects, the solid pharmaceutical composition includes a surfactant. A surfactant may also be added to reduce aggregation of the compound and/or to minimize the formation of particulates in the formulation and/or to reduce adsorption. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.01% w/v.

In various aspects, the solid pharmaceutical composition may also include various materials that modify the physical form of the dosage unit. For example, the composition may include materials that form a coating shell around the active ingredients. The materials that form the coating shell are typically inert, and may be selected from, for example, sugar, shellac, polymers, plasticizers, pigments, or other coating agents.

In various aspects, the solid pharmaceutical composition is in the form of a solid powder and is encased within a suitable capsule, such as a gelatin capsule. The amount of drug contained within such a capsule can vary depending upon recognized factors, such as frequency and duration of therapy, dose intervals, excretion rate, the recipient's age, body weight, sex, diet, medical history and general state (e.g., health), the severity of the disease, and/or the size, malignancy and invasiveness of a tumor to be treated The compound is thus in a form suitable, and administered at a dosage sufficient to, achieve a desired therapeutic or prophylactic effect.

Administration of the solid dosage form of ATR-101 is typically in the form of one or more dosage units wherein, for example, a single tablet or capsule is a single dosage unit. The dosage amount and frequency are selected to create a therapeutically effective level of the agent. For example, when administered with meals, such as breakfast, lunch or dinner, frequency may be three times daily (once with each meal). Alternatively, administration with food may be once daily; twice daily, three times daily, or four times daily; alternating days; every third day; or 2, 3, 4, 5, or 6 times per week; weekly; twice a month; monthly or more or less frequently, as necessary, depending on the response or condition and the recipient tolerance of the therapy. Maintenance dosages over a longer period of time, such as 4, 5, 6, 7, 8, 10 or 12 weeks or longer, are contemplated, and dosages may be adjusted as necessary. Similarly, intermittent dosing, where dosing is stopped for a period of time and then restarted, is also contemplated. The progress of the therapy may be monitored by conventional techniques and assays, such as by monitoring tumor size and/or cortisol or other adrenal hormone levels, and is within the skill in the art.

In one embodiment, oral administration of a plurality of dosage units is contemplated once daily, twice daily, three times daily or four times daily. Depending up the amount of ATR-101 contained within, for example, a single tablet, a plurality of unit dosage forms may range from 2 to 30 tablets administered once daily, twice daily, three times daily or four times daily. In one embodiment, and depending upon exposure as discussed below, administration may be 4-24 tablets twice daily, or 6-12 tablets twice daily.

In some embodiments, the solid drug form is administered at a dosage, on a daily basis, of from about 0.1 mg/kg to about 200 mg/kg. Suitable dosages include, but are not limited to, from 0.5 mg/kg to 150 mg/kg, from 0.75 mg/kg to 100 mg/kg, from 1 mg/kg to 50 mg/kg, from 2 mg/kg to 40 and from 3 mg/kg to 35 mg/kg. Suitable dosages also include from 1 mg/kg to 5 mg/kg and from 2 mg/kg to 4 mg/kg, as well as from 10 mg/kg to 50 mg/kg and from 20 mg/kg to 40 mg/kg.

Methods of the present invention include treatment of a disorder by administrating to a subject in need thereof a therapeutically effective amount of the solid pharmaceutical composition comprising ATR-101. In various embodiments, the methods comprise co-administration to a subject in need thereof a therapeutically effective amount of the solid pharmaceutical composition in combination with food. In various aspects, methods are also provided for slowing or stopping progression of disorder, preventing a disorder, or reversing a disorder.

In one embodiment, the disorder is a disorder associated with aberrant adrenocortical cellular activity, and the method comprises administration to a subject in need thereof a therapeutically effective amount of the solid pharmaceutical composition. In various aspects, methods are also provided for slowing or stopping progression of a disorder associated with aberrant adrenocortical cellular activity. In various aspects, methods are also provided for preventing a disorder associated with aberrant adrenocortical cellular activity. In various aspects, methods are also provided for reversing a disorder associated with aberrant adrenocortical cellular activity.

In this embodiment, methods according to the present invention include treating: increased hormone or hormone precursor production, benign adenoma, adrenocortical carcinoma (ACC), metastatic adrenocortical carcinoma, congenital adrenal hyperplasia, Cushing's syndrome, excess cortisol production, symptoms associated with excess cortisol production, hyperaldosteronism, Conn syndrome, unilateral aldosterone-producing adenoma, bilateral adrenal hyperplasia (or idiopathic hyperaldosteronism (IHA)), primary adrenal hyperplasia, glucocorticoid-remediable aldosteronism (GRA) and/or 21-hydroxylase deficiency. Such methods involve administration to a subject in need thereof a therapeutically effective amount of the solid pharmaceutical composition.

In other embodiments, methods according to the present invention include reducing adrenocortical tumor size, and/or inhibiting aberrant adrenal hormone production in a patient, by administration to a subject in need thereof a therapeutically effective amount of the solid pharmaceutical composition.

Methods according to the present invention also include slowing or stopping progression of: increased hormone production, benign adenoma, adrenocortical carcinoma, metastatic adrenocortical carcinoma, Cushing's syndrome, excess cortisol production, symptoms associated with excess cortisol production, congenital adrenal hyperplasia, hyperaldosteronism, Conn syndrome, unilateral aldosterone-producing adenoma, bilateral adrenal hyperplasia (or idiopathic hyperaldosteronism (IHA)), renin-responsive adenoma, primary adrenal hyperplasia, glucocorticoid-remediable aldosteronism (GRA), 21-hydroxylase deficiency. Such methods involve administration to a subject in need thereof a therapeutically effective amount of the solid pharmaceutical composition.

In another embodiment, the disorder is a disorder that would benefit from inhibition of ACAT, and the method comprises administration to a subject in need thereof a therapeutically effective amount of the solid pharmaceutical composition. In various aspects, methods are also provided for slowing or stopping progression of a disorder that would benefit from inhibition of ACAT. In various aspects, methods are also provided for preventing a disorder that would benefit from inhibition of ACAT. In various aspects, methods are also provided for reversing a disorder that would benefit from inhibition ACAT. In this embodiment, methods according to the present invention include treating prostate cancer and ovarian cancer.

Methods according to the present invention also include co-therapy by administration of a second therapeutic agent, including known chemotherapeutics, targeting agents, adrenalysis agents, metformin, everolimus, and/or IGF1R antagonist. In one embodiment, co-administration further comprises administering mitotane. Examples of suitable chemotherapeutic and radio therapeutic agents include, but are not limited to: an anti-metabolite; a DNA-damaging agent; a cytokine useful as a chemotherapeutic agent; a covalent DNA-binding drug; a topoisomerase inhibitor; an anti-mitotic agent; an anti-tumor antibiotic; a differentiation agent; an alkylating agent; a methylating agent; a hormone or hormone antagonist; a nitrogen mustard; a radio sensitizer; a photosensitizer; a radiation source, optionally together with a radio sensitizer or photosensitizer; or other commonly used therapeutic agents.

In another embodiment, kits are provided for co-administration of ATR-101 with food to increase exposure of ATR-101 upon oral administration. Such kits comprise a plurality of oral unit dosage forms of ATR-101 in combination with instructions for co-administration with food.

Various alternative embodiments and examples of the invention are described herein. These embodiments and examples are illustrative and should not be construed as limiting the scope of the invention.

### EXAMPLES

### Example 1

### Synthesis of ATR-101

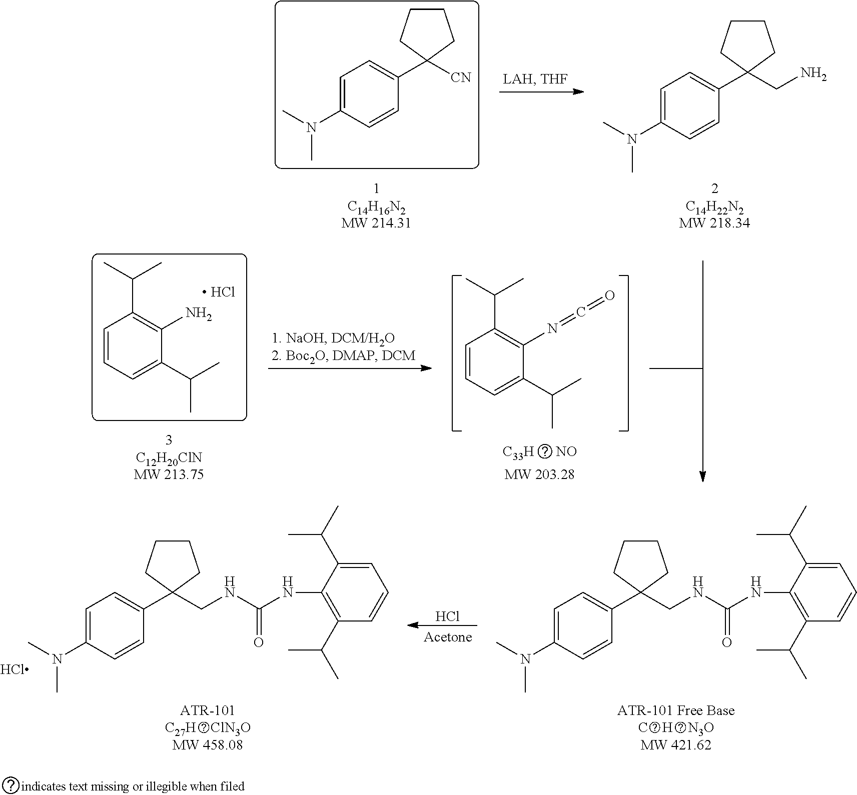

Step 1: Preparation of Primary Amine 2 from the Nitrile 1

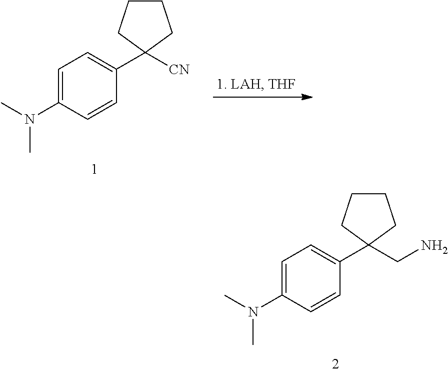 [SMILES: CN(C)c1ccc(C2(C#N)CCCC2)cc1 ; CN(C)c1ccc(C2(CN)CCCC2)cc1]

Tetrahydrofuran (THF) and Compound 1 are charged to a reactor vessel and a lithium aluminum hydride (LAH) solution in THF is added slowly. After the addition, the reaction mixture is warmed to 45° C. and stirred until in-process HPLC analysis indicates that the reaction is complete. The reaction mixture is cooled to between 0 and 10° C. and aqueous NaOH is added slowly while controlling the temperature to between 0 and 10° C. The mixture is then warmed to between 20 and 25° C. and any inorganic salts removed by filtration. The solids are then washed with additional THF.

The filtrate is distilled under vacuum. Acetonitrile (MeCN) is added and the distillation continued to reduce the total volume. H₂O is added and the solution is cooled to 20° C., and seeded if necessary. Additional water is added to the slurry and cooled to between 0 and 5° C. and filtered. The crystallization vessel and filter cake is washed with MeCN and water (1:2 mixture) and dried under vacuum between 40 to 45° C. to produce Compound 2. Typical yield: 85%.

### Step 2: Preparation of ATR-101 Free Base

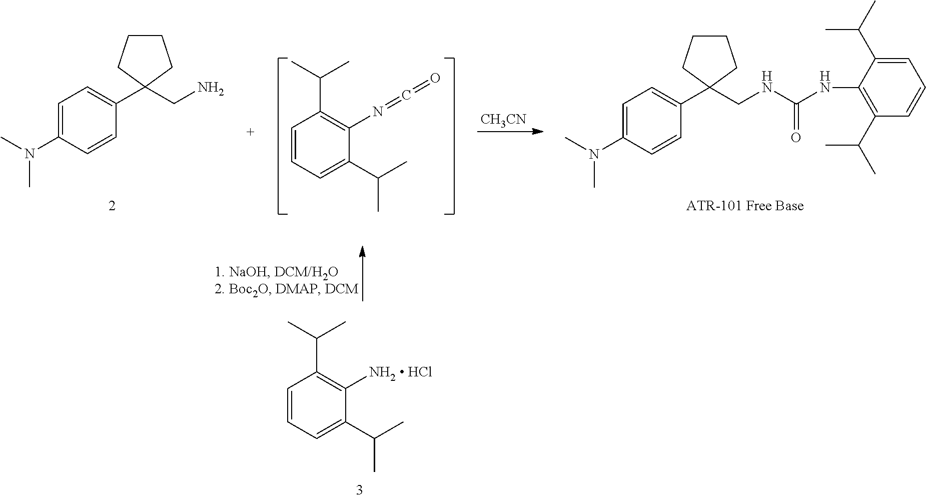

2,6-Diisopropyl aniline hydrochloride (Compound 3) is converted to the corresponding free base by stirring in a mixture of dichloromethane (DCM) and 10% aqueous NaOH. The organic phase is separated and washed with water. The DCM solution containing the aniline free base is concentrated by distillation.

4-dimethylaminopyridine (DMAP) and DCM are charged to a separate reaction vessel. The mixture is cooled and a solution of di-tert-butyl dicarbonate (Boc₂O) in DCM is slowly added while the temperature is maintained between 0 and 5° C. The aniline free base solution is then slowly added to the reaction vessel. A complete conversion of aniline to the isocyanate is verified by in-process HPLC analysis.

Compound 2 and MeCN are charged to a separate vessel and this solution is cooled to between 0 and 5° C. The isocyanate intermediate solution (prepared above) is slowly added while the temperature is maintained between 0 and 5° C., and stirred until in-process HPLC indicates that the reaction is complete.

The reaction mixture is distilled under vacuum, and isopropyl alcohol (IPA) is added and the distillation is continued. The resulting solution is cooled and seeded, if necessary. After crystallization occurs, water is added and the mixture is cooled to between 0 and 5° C., and filtered. The crystallization vessel and filter cake is washed with isopropanol:water (1:1) and the product cake is dried under vacuum to yield ATR-101 as the free base. Typical yield: 89%

### Step 3: Preparation of Solid Drug Form of ATR-101

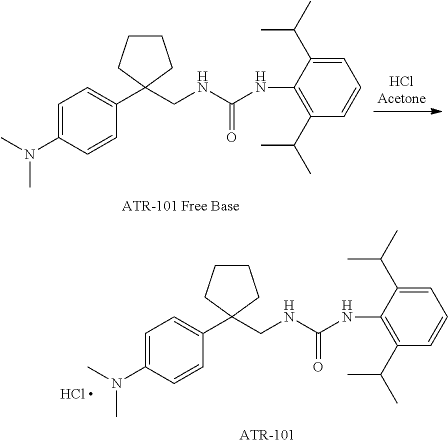 [SMILES: CC(C)c1cccc(C(C)C)c1NC(=O)NCC1(c2ccc(N(C)C)cc2)CCCC1 ; CC(C)c1cccc(C(C)C)c1NC(=O)NCC1(c2ccc(N(C)C)cc2)CCCC1 ; "ATR-101"]

The ATR-101 free base is dissolved in acetone and filtered to remove particulates. Additional acetone is used to rinse the dissolution vessel and filter. Concentrated hydrochloric acid (HCl) is added while maintaining the reaction at room temperature. The resultant slurry is filtered and the cake is washed with acetone. The resulting solid is dried under vacuum between 40 and 45° C. to obtain the solid drug form of ATR-101. Typical yield: 70-80%.

### Example 2

### Preparation of Tablet Containing ATR-101

Tablets containing 500 mg ATR-101 (as the free base) may be prepared according to the procedure set forth below, and the make-up of exemplary tablets are listed in Tables 1 and 2.

### A. High Shear Wet Granulation

1. ATR-101 is weighed.
2. Mannitol is weighed and passed through a No. 30 mesh screen.
3. Microcrystalline cellulose, croscarmellose, and pregelatinized starch are weighed and passed through a No. 30 mesh screen.
4. A pre-granulation mix of the intragranular excipients (components 2 and 3 above) and ATR-101 is prepared using a V-blender.
5. Hypromellose is weighed and dissolved in water under stirring.
6. The pregranulation mix is wet granulated with the hypromellose solution using a high shear granulator.
7 The wet granules are deagglomerated by passing through a No. 7 mesh screen. The granules are then dried in a fluid bed drier. The drying endpoint is determined by loss-on-drying (LOD).

### B. Addition of Extragranular Excipients

1. The dried granules are passed through a No. 20 mesh screen and reintroduced into the V-blender.
2. Croscarmellose and magnesium stearate were are through a No. 30 mesh screen and added to the V-blender.
3. The dried granules and extragranular excipients are then mixed.

### C. Compression

1. Tablet cores are compressed using a gravity fed rotary tablet press.

### D. Film Coating

1. Core tablets maybe coated using a suitable coating solution.

TABLE 1
Ingredients of Representative 500 mg ATR-101 Tablet

| Ingredient | mg/tablet | |
|---|---|---|
| ATR-101 (as free base) | 500 | mg |
| Mannitol | 150 | mg |
| Microcrystalline Cellulose Type 101 | 170 | mg |
| Croscarmellose sodium | 20 | mg |
| Pregelatinized starch | 50 | mg |
| Hypromellose | 30 | mg |
| Microcrystalline Cellulose Type 102 | 50 | mg |
| Magnesium Stearate | 20 | mg |

| | |
|---|---|
| Water (removed during processing) | — |

The following coated tablet was made in a similar manner as described above.

TABLE 2
Ingredients of Representative 500 mg ATR-101 Tablet

| Ingredient | mg/tablet | |
|---|---|---|
| ATR-101 (as free base) | 500 | mg |
| Mannitol | 54.3 | mg |
| Microcrystalline Cellulose Type 101 | 54.3 | mg |
| Croscarmellose sodium | 36.2 | mg |
| Pregelatinized starch | 14.5 | mg |
| Hypromellose | 14.5 | mg |
| Magnesium Stearate | 7.2 | mg |
| Opadry II White | 21.7 | mg |

| | |
|---|---|
| Water (removed during processing) | — |

### Example 3

### Enhanced Bioavailability with Food Co-Administration

To evaluate the effects of food and of an acidic beverage on bioavailability of ATR-101 tablets prepared according to Example 2 (see Table 2), an open-label, randomized, 3-period, 3-way crossover, 3-sequence study was carried out with fourteen healthy, adult, non-tobacco using male and female (non-childbearing potential only) human subjects.

Three different treatments A, B, and C were administered as follows:

Treatment A: 500 mg ATR-101 (1×500 mg tablet) administered with water at Hour 0 on Day 1 following an overnight fast.

Treatment B: 500 mg ATR-101 (1×500 mg tablet) administered with water at Hour 0, 30 minutes after the start of a high-fat breakfast, on Day 1.

Treatment C: 500 mg ATR-101 (1×500 mg tablet) administered with Coca-Cola® Classic at Hour 0 on Day 1 following an overnight fast.

On Day 1 of Period 1, subjects were randomized to one of 3 treatment sequences: ABC, BCA, or CBA. For Treatments A and B, ATR-101 was administered orally with approximately 240 mL of water. For Treatment C, ATR-101 was administered orally with approximately 240 mL of Coca-Cola® Classic. Water (except water provided with dosing for Treatments A and B) was restricted 1 hour prior to and 1 hour after each study drug administration, but was allowed ad libitum at all other times. Other fluids were given as part of the standard meals (including the breakfast administered prior to study drug dosing for Treatment B) and/or snacks, but were restricted at all other times (except for the Coca-Cola® Classic beverage provided with dosing in Treatment C) throughout the confinement period.

For Treatments A and C, subjects fasted overnight for at least 10 hours prior to study drug administration and continued the fast for at least 4 hours post-dose.

For Treatment B, subjects were required to fast overnight for at least 10 hours until 30 minutes prior to study drug administration, when they were given breakfast which was entirely consumed within 30 minutes. A representative breakfast includes 2 slices of buttered toast, 2 fried eggs, 2 strips of bacon, 4 oz of hash brown potatoes, and 240 mL of whole milk. Subjects did not eat for at least 4 hours following dosing.

On Day 1 of each period, a standard lunch and dinner was provided at approximately 4 and 9 hours post-dose, respectively, and a snack was also offered at approximately 12 hours post-dose. On other days when confined in the CRU, standard meals and/or snacks were provided at appropriate times. When confined, subjects were required to fast from all food and drink except water between meals and snacks served at the clinical research unit.

Each meal and/or snack served at the clinical research unit was standardized and was similar in caloric content and composition (except for the meal served as part of Treatment B) and was taken at approximately the same time in each period.

For all subjects, blood samples for the determination of ATR-101 plasma concentration were collected at pre-dose and 0.5, 1, 1.5, 2, 2.5, 3, 3.5, 4, 5, 6, 8, 12, 16, 24, 36, and 48 hours post-dose. PK parameters for plasma ATR-101 were calculated as follows:
AUC₀₋ₜ: The area under the plasma concentration-time curve, from time 0 to the last measurable non-zero concentration, was calculated by the linear trapezoidal method.
AUC_{0-inf}: The area under the plasma concentration-time curve from time 0 extrapolated to infinity. AUC_{0-inf} was calculated as the sum of AUC₀₋ₜ plus the ratio of the last measurable plasma concentration to the elimination rate constant.
AUC_{%extrap}: Percent of AUC_{0-inf} extrapolated, represented as

$$( 1 - \frac{{AUC}_{0 - t}}{{AUC}_{0 - inf}} ) \times 100.$$

Cₘₐₓ: Maximum observed concentration.
tₘₐₓ: Time to reach Cₘₐₓ. If the maximum value occurred at more than one time point, tₘₐₓ was defined as the first time point with this value.
kₑ₁: Apparent first-order terminal elimination rate constant calculated from a semi-log plot of the plasma concentration versus time curve. The parameter was calculated by linear least-squares regression analysis using the maximum number of points in the terminal log-linear phase (e.g., three or more non-zero plasma concentrations).
t^{1/2}: Apparent first-order terminal elimination half-life was calculated as 0.693/kₑ₁.

No value for kₑ₁, AUC_{0-inf}, or t_{1/2} was reported for cases that did not exhibit a terminal log-linear phase in the concentration versus time profile. No PK parameters were calculated for subjects with detectable concentrations for 2 or fewer consecutive time points.

TABLE 3
Individual Plasma ATR-101 Concentrations (ng/mL) and Summary Statistics Following
Single Oral Administration of 500 mg ATR-101
Treatment A—Fasting with Water

| | | | Parameters | | | | | | |
|---|---|---|---|---|---|---|---|---|---|
| | | | AUC₀₋ₜ | AUC_{0-inf} | AUC_{% extrap} | Cₘₐₓ | | kₑₗ | |
| Subject No. | Treatment Sequence | Study Period | $\frac{ng \times hr}{mL}$ | $\frac{ng \times hr}{mL}$ | $\frac{ng \times hr}{mL}$ | $\frac{ng}{mL}$ | tₘₐₓ hr | $\frac{1}{hr}$ | t_{1/2} hr |
| 1 | CAB | 2 | 3880 | 4370 | 11.2 | 842 | 2.00 | 0.0318 | 21.8 |
| 2 | BCA | 3 | 2450 | 2740 | 10.6 | 509 | 2.5 | 0.0378 | 18.3 |
| 3 | ABC | 1 | 3180 | 3400 | 6.68 | 620 | 2.00 | 0.0414 | 16.8 |
| 4 | ABC | 1 | 3810 | 4290 | 11.2 | 807 | 1.50 | 0.0347 | 20.0 |
| 5 | CAB | 2 | 2490 | 2630 | 5.32 | 542 | 2.00 | 0.0456 | 15.2 |
| 6 | BCA | 3 | 840 | * | * | 26.1 | 12.0 | * | * |
| 7 | ABC | 1 | 2200 | 2430 | 9.43 | 612 | 1.50 | 0.0380 | 18.2 |
| 8 | CAB | 2 | 4860 | 5050 | 3.72 | 728 | 1.50 | 0.0612 | 11.3 |
| 9 | BCA | 3 | 2330 | 2560 | 8.82 | 539 | 1.50 | 0.0404 | 17.2 |
| 10 | BCA | 3 | 1520 | * | * | 306 | 2.00 | * | * |
| 11 | ABC | 1 | 4100 | 4700 | 12.7 | 526 | 1.5 | 0.0317 | 21.9 |
| 12 | CAB | 2 | 2620 | 2740 | 4.61 | 486 | 1.50 | 0.0531 | 13.1 |
| 14 | CAB | 2 | 4400 | 4940 | 10.8 | 933 | 3.00 | 0.0305 | 22.7 |

| | | | | | | | |
|---|---|---|---|---|---|---|---|
| N | 13 | 11 | 11 | 13 | 13 | 11 | 11 |
| Mean | 2970 | 3620 | 8.64 | 575 | NA | 0.0406 | 17.9 |
| SD | 1180 | 1050 | 3.06 | 236 | NA | 0.00957 | 3.68 |
| CV (%) | 39.7 | 29.0 | 35.5 | 41.1 | NA | 23.6 | 20.6 |
| Minimum | 840 | 2430 | 3.72 | 26.1 | 1.50 | 0.0305 | 11.3 |
| Median | 2620 | 3400 | 9.43 | 542 | 2.00 | 0.0380 | 18.2 |
| Maximum | 4860 | 5050 | 12.7 | 933 | 12.0 | 0.0612 | 22.7 |
| Geometric Mean | 2710 | 3480 | NA | 469 | NA | NA | NA |
| Geometric CV (%) | 51.4 | 30.0 | NA | 114.4 | NA | NA | NA |

Subject 13 was excluded from PK parameter calculations because predose concentration was >5% of Cₘₐₓ.
NA = Not Applicable
* = Value missing or not reportable

TABLE 4
Individual Plasma ATR-101 Concentrations (ng/mL) and Summary Statistics Following
Single Oral Administration of 500 mg ATR-101
Treatment B—Fed with Water

| | | | Parameters | | | | | | |
|---|---|---|---|---|---|---|---|---|---|
| | | | AUC₀₋ₜ | AUC_{0-inf} | AUC_{% extrap} | Cₘₐₓ | | kₑₗ | |
| Subject No. | Treatment Sequence | Study Period | $\frac{ng \times hr}{mL}$ | $\frac{ng \times hr}{mL}$ | $\frac{ng \times hr}{mL}$ | $\frac{ng}{mL}$ | tₘₐₓ hr | $\frac{1}{hr}$ | t_{1/2} hr |
| 1 | CAB | 2 | 6570 | 7210 | 8.80 | 993 | 3.57 | 0.0374 | 18.6 |
| 2 | BCA | 3 | 3520 | 3790 | 7.07 | 1040 | 1.50 | 0.0359 | 19.3 |
| 3 | ABC | 1 | 5930 | 6400 | 7.35 | 1620 | 1.50 | 0.0332 | 20.9 |
| 4 | ABC | 1 | 13200 | 14000 | 5.87 | 2060 | 2.50 | 0.0410 | 16.9 |
| 5 | CAB | 2 | 5280 | 5390 | 2.07 | 1120 | 3.00 | 0.0603 | 11.5 |
| 6 | BCA | 3 | 4120 | 4540 | 9.33 | 848 | 3.00 | 0.0276 | 25.1 |
| 7 | ABC | 1 | 6890 | 7640 | 9.87 | 1340 | 2.00 | 0.0249 | 27.8 |
| 8 | CAB | 2 | 8430 | 9000 | 6.38 | 2000 | 1.50 | 0.0385 | 18.0 |
| 9 | BCA | 3 | 6810 | 7660 | 1.1 | 1210 | 2.50 | 0.0278 | 24.9 |
| 10 | BCA | 3 | 6290 | 7670 | 17.9 | 931 | 5.00 | 0.0226 | 30.7 |
| 11 | ABC | 1 | 5420 | 5830 | 7.19 | 719 | 4.01 | 0.0398 | 17.4 |
| 12 | CAB | 2 | 5310 | 5820 | 8.76 | 1180 | 5.00 | 0.0290 | 23.9 |
| 13 | BCA | 1 | 4740 | 4900 | 3.35 | 759 | 5.00 | 0.0526 | 13.2 |
| 14 | CAB | 2 | 9300 | 9990 | 6.89 | 1690 | 2.50 | 0.0392 | 17.7 |

| | | | | | | | |
|---|---|---|---|---|---|---|---|
| N | 14 | 14 | 14 | 14 | 14 | 14 | 14 |
| Mean | 6560 | 7130 | 8.00 | 1250 | NA | 0.0364 | 20.4 |
| SD | 2460 | 2630 | 3.74 | 437 | NA | 0.0104 | 5.45 |
| CV (%) | 37.5 | 36.8 | 46.7 | 34.9 | NA | 28.7 | 26.7 |
| Minimum | 3520 | 3790 | 2.07 | 719 | 1.50 | 0.0226 | 11.5 |
| Median | 6110 | 6810 | 7.27 | 1150 | 2.75 | 0.0366 | 18.9 |
| Maximum | 13200 | 14000 | 17.9 | 2060 | 5.00 | 0.0603 | 30.7 |
| Geometric Mean | 6200 | 6740 | NA | 1180 | NA | NA | NA |
| Geometric CV (%) | 34.8 | 35.1 | NA | 34.8 | NA | NA | NA |

NA = Not Applicable

TABLE 5
Individual Plasma ATR-101 Concentrations (ng/mL) and Summary Statistics Following
Single Oral Administration of 500 mg ATR-101
Treatment C—Fasting with Acidic Beverage

| | | | Parameters | | | | | | |
|---|---|---|---|---|---|---|---|---|---|
| | | | AUC₀₋ₜ | AUC_{0-inf} | AUC_{% extrap} | Cₘₐₓ | | kₑₗ | |
| Subject No. | Treatment Sequence | Study Period | $\frac{ng \times hr}{mL}$ | $\frac{ng \times hr}{mL}$ | $\frac{ng \times hr}{mL}$ | $\frac{ng}{mL}$ | tₘₐₓ hr | $\frac{1}{hr}$ | t_{1/2} hr |
| 1 | CAB | 2 | 4300 | 5020 | 14.2 | 893 | 2.50 | 0.0256 | 27.1 |
| 2 | BCA | 3 | 572 | 728 | 21.5 | 19.8 | 6.00 | 0.0397 | 17.5 |
| 3 | ABC | 1 | 3800 | 3990 | 4.67 | 734 | 2.00 | 0.0501 | 13.8 |
| 4 | ABC | 1 | 5100 | 5760 | 11.3 | 1030 | 2.00 | 0.0380 | 8.2 |
| 5 | CAB | 2 | 2510 | 2610 | 3.84 | 438 | 2.00 | 0.0496 | 14.0 |
| 6 | BCA | 3 | 4940 | 5420 | 8.70 | 948 | 2.00 | 0.0371 | 18.7 |
| 7 | ABC | 3 | 4110 | 4530 | 9.20 | 645 | 3.00 | 0.0345 | 20.1 |
| 8 | CAB | 2 | 6430 | 6970 | 7.61 | 952 | 2.00 | 0.0353 | 9.7 |
| 10 | BCA | 3 | 2360 | 2880 | 18.3 | 461 | 1.50 | 0.0249 | 7.9 |
| 11 | ABC | 1 | 3660 | * | * | 602 | 2.50 | * | * |
| 12 | CAB | 2 | 3400 | 3670 | 7.31 | 666 | 2.00 | 0.0433 | 16.0 |
| 13 | BCA | 1 | 2250 | 2670 | 15.6 | 525 | 2.50 | 0.0258 | 26.9 |
| 14 | CAB | 2 | 6310 | 6680 | 5.52 | 1550 | 3.00 | 0.0488 | 14.2 |

| | | | | | | | |
|---|---|---|---|---|---|---|---|
| N | 13 | 13 | 12 | 13 | 13 | 12 | 12 |
| Mean | 3830 | 4240 | 10.6 | 728 | NA | 0.0377 | 19.5 |
| SD | 1670 | 1850 | 5.62 | 367 | NA | 0.00916 | 5.15 |
| CV (%) | 43.6 | 43.5 | 52.8 | 50.4 | NA | 24.3 | 26.4 |
| Minimum | 572 | 728 | 3.84 | 19.8 | 1.50 | 0.0249 | 13.8 |
| Median | 3800 | 4260 | 8.95 | 666 | 2.00 | 0.0376 | 18.4 |
| Maximum | 6430 | 6970 | 21.5 | 1550 | 6.00 | 0.0501 | 27.9 |
| Geometric Mean | 3340 | 3720 | NA | 558 | NA | NA | NA |
| Geometric CV (%) | 69.9 | 67.8 | NA | 145.0 | NA | NA | NA |

Subject 9 was excluded from PK parameter calculations because predose concentration was >5% of Cₘₐₓ.
NA = Not Applicable
* = Value missing or not reportable

TABLE 6
Ratios of Plasma ATR-101 AUC₀₋ₜ
Following Single Oral Administration of 500 mg ATR-101

| | | AUC₀₋ₜ | | | | | |
|---|---|---|---|---|---|---|---|
| | | Treatment | Treatment | Treatment | | | |
| | | A | B | C | Ratio | Ratio | Ratio |
| Subject No. | Treatment Sequence | $\frac{ng \times hr}{mL}$ | $\frac{ng \times hr}{mL}$ | $\frac{ng \times hr}{mL}$ | B/A % | C/A % | B/C % |
| 1 | CAB | 3880 | 6570 | 4300 | 169 | 111 | 153 |
| 2 | BCA | 2450 | 3520 | 572 | 144 | 23.3 | 615 |
| 3 | ABC | 3180 | 5930 | 3800 | 187 | 120 | 156 |
| 4 | ABC | 3810 | 13200 | 5100 | 346 | 134 | 258 |
| 5 | CAB | 2490 | 5280 | 2510 | 212 | 101 | 210 |
| 6 | BCA | 840 | 4120 | 4940 | 490 | 588 | 83.2 |
| 7 | ABC | 2200 | 6890 | 4110 | 313 | 187 | 168 |
| 8 | CAB | 4860 | 8430 | 6430 | 173 | 132 | 131 |
| 9 | BCA | 2330 | 6810 | * | 292 | * | * |
| 10 | BCA | 1520 | 6290 | 2360 | 415 | 155 | 267 |
| 11 | ABC | 4100 | 5420 | 3660 | 132 | 89.3 | 148 |
| 12 | CAB | 2620 | 5310 | 3400 | 203 | 130 | 156 |
| 13 | BCA | * | 4740 | 2250 | * | * | 211 |
| 14 | CAB | 4400 | 9300 | 6310 | 211 | 143 | 147 |

| | | | | | | |
|---|---|---|---|---|---|---|
| N | 13 | 14 | 13 | 13 | 12 | 13 |
| Mean | 2970 | 6560 | 3830 | 253 | 160 | 208 |
| SD | 1180 | 2460 | 1670 | 110 | 141 | 131 |
| CV (%) | 39.7 | 37.5 | 43.6 | 43.6 | 88.3 | 63.7 |
| Minimum | 840 | 3520 | 572 | 132 | 23.3 | 83.2 |
| Median | 2620 | 6110 | 3800 | 211 | 131 | 156 |
| Maximum | 4860 | 13200 | 6430 | 490 | 588 | 615 |
| Geometric Mean | 2710 | 6200 | 3340 | 233 | 126 | 184 |
| Geometric CV (%) | 51.4 | 34.8 | 69.9 | 42.7 | 81.7 | 50.1 |

Treatment A: 500 mg ATR-101 with water (fasting)
Treatment B: 500 mg ATR-101 with water (fed)
Treatment C: 500 mg ATR-101 with acidic beverage (fasting)
Subject 9 Treatment C and Subject 13 Treatment A were excluded from PK parameter calculations because predose concentration was >5% of Cₘₐₓ.
% Ratio = 100 × (Treatment at numerator/ Treatment at denominator)
* = Value missing or not reportable

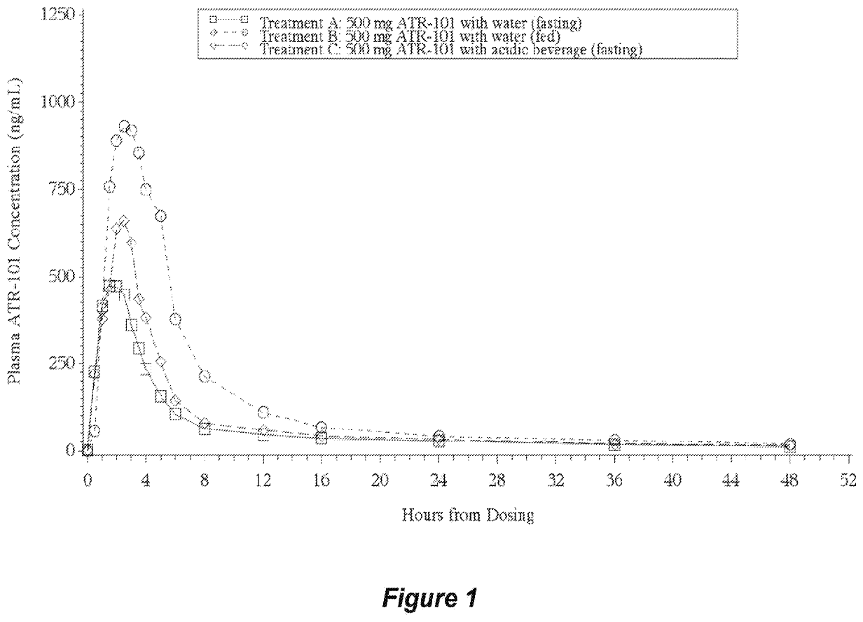

FIG. 1 is the mean plasma concentration linear time curves for Treatments A, B, and C. ATR-101 administered in a fed state (Treatment B) showed a 153% increase in systemic exposure compared to a fasting state administered with water (Treatment A) and a 108% increase in systemic exposure compared to a fasting state administered with an acidic beverage (Treatment C). This data demonstrates the significant benefit of a fed-state in the oral administration of ATR-101.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way.

The disclosure of U.S. provisional patent application Ser. No. 62/191,195, filed Jul. 10, 2015, is incorporated herein in its entirety.

Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A method of increasing the bioavailability of ATR-101 comprising orally administering to a subject in need thereof ATR-101 in unit dosage form at or near the time of oral administration of food.

2. The method of claim 1, wherein the ATR-101 in unit dosage form is administered at the same time or within about 30 minutes after the oral administration of food.

3. The method of claim 1, wherein the food is a high fat, high calorie meal.

4. The method of claim 1, wherein the mean maximum plasma concentration (Cₘₐₓ) of ATR-101 in the subject in need thereof is increased when the unit dosage form of ATR-101 is administered with a meal, compared to when the unit dosage form of ATR-101 is administered under fasting conditions.

5. The method of claim 4, wherein Cₘₐₓ increases by at least about 50%.

6. The method of claim 4, wherein Cₘₐₓ increases by at least about 100%.

7. The method of claim 1, wherein the area under the plasma concentration time curve (AUC₀₋ₜ) of ATR- in the subject in need thereof is increased when the unit dosage form of ATR-101 is administered with a meal, compared to when ATR-101 is administered under fasting conditions.

8. The method of claim 7, wherein AUC₀₋ₜ increases by at least about 50%.

9. The method of claim 7, wherein AUC₀₋ₜ increases by at least about 100%.

10. The method of claim 1, wherein the subject has a non-cancerous disorder.

11. The method of claim 10, wherein the subject has a non-cancerous endocrine disorder.

12. The method of claim 10, wherein the subject has Cushing's syndrome.

13. The method of claim 10, wherein the subject has congenital adrenal hyperplasia.

14. The method of claim 1, wherein the subject has a cancerous disorder.

15. The method of claim 14, wherein the subject has ACC.

16. The method of claim 14, wherein the subject has prostate cancer.

17. A solid pharmaceutical composition in a unit dosage form suitable for oral administration, comprising N-(2,6-bis(1-methylethyl)phenyl)-N′-((1-(4-(dimethylamino)phenyl)-cyclopentyl)methyl)urea hydrochloride (ATR-101) in combination with one or more pharmaceutically acceptable carriers or excipients, wherein ATR-101 is present in the unit dosage form at a level ranging from about 250-750 mg as measured as the free base form of ATR-101.

18.-33. (canceled)

34. A method of administering a solid pharmaceutical composition of claim 17, comprising orally administering to a subject in need thereof ATR-101 in unit dosage form.

35.-50. (canceled)

51. A kit comprising a plurality of oral unit dosage forms of the solid pharmaceutical composition of claim 17 in combination with instructions for co-administration with food at or near the time of oral administration of ATR-101 in unit dosage form.
